(19) United States
(12) Patent Application Publication
(10) Pub. No.: US 2009/0098196 A1
(43) Pub. Date: Apr. 16, 2009

(54) LIPID CARRIER COMPOSITIONS WITH REDUCED POLY-DISPERSITY

(76) Inventors: Paul Tardi, Surrey (CA); Lawrence Mayer, North Vancouver (CA); Donna Cabral-Lilly, Pennington, NJ (US)

Correspondence Address: MORRISON & FOERSTER LLP, 12531 HIGH BLUFF DRIVE, SUITE 100, SAN DIEGO, CA, 92130-2040, US

(21) Appl. No.: 11/813,958

(22) PCT Filed: Jan. 26, 2006
(86) PCT No.: PCT/US06/02782

§ 371 Date: Apr. 7, 2008

Related U.S. Application Data
(60) Provisional application No. 60/647,694, filed on Jan. 26, 2005.

(51) Int. Cl. A61K 9/127

(52) U.S. Cl. 424/450

(57) ABSTRACT

The invention relates to a method for reducing the polydispersity of a population of gel-phase lipid-based delivery vehicles.

### TECHNICAL FIELD

The invention relates to a method to improve the homogeneity of a population of lipid-based carriers and to delivery vehicle compositions formed thereby. More particularly, the invention concerns a filter-extrusion method which results in reduced polydispersity of a population of gel or solid-phase lipid-based delivery vehicles.

### BACKGROUND ART

Liposomes and other lipid-based carrier systems have been extensively developed and analyzed for their ability to improve the therapeutic index of drugs by altering their pharmacokinetics and tissue distribution. This approach is aimed at reducing exposure of healthy tissues to therapeutic agents while increasing drug delivery to a diseased site. In order for the therapeutic effectiveness of liposome-encapsulated agents to be realized, the agents must be well-retained within a liposome after intravenous administration and the liposomes must have a sufficient circulation lifetime to permit the desired drug delivery.

It has long been established that incorporation of membrane-rigidifying agents such as cholesterol into a liposomal membrane enhances the circulation lifetime of the liposome as well as the retention of encapsulated drugs. Inclusion of cholesterol in liposomal membranes has been shown to reduce release of drug after intravenous administration (for example, see: U.S. Pat. Nos. 4,756,910, 5,077,056, and 5,225,212; Kirby, C., et al., Biochem. J. (1980) 186:591-598; and, Ogihara-Umeda, I., et al., Eur. J. Nucl. Med. (1989) 15:612-617). Generally, cholesterol increases bilayer thickness and order while decreasing membrane permeability, protein interactions, and lipoprotein destabilization of the liposome. Conventional approaches to liposome formulation dictate inclusion of substantial amounts (e.g., greater than 30 mol %) of cholesterol or equivalent membrane-rigidifying agents.

More recently, researchers have reported the benefits of utilizing “low-cholesterol” (typically less than 25 mol %) liposomes (see WO 03/041681 and WO 03/041682). Contrary to the general teachings of the art, inclusion of low levels of cholesterol results in enhanced drug retention of certain drugs as well as increased circulation longevity of certain liposomes.

Because of the nature of lipid-based vesicles and the methods used to make them, the initial hydration of the lipid films results in a polydisperse (i.e., heterogeneous in size) population of, for example, multi-lamellar vesicles (MLV's), many of which are greater than about 1 micron. For parenteral administration, delivery vehicles are preferably about 50-200 nm in diameter. In this size range, conventional lipid-based delivery vehicles are successfully filter sterilized with filters whose pore sizes are about 0.2 microns. Filter sterilization is a preferred method for sterilizing liposome solutions since most liposomes can not stably withstand autoclaving or high-energy radiation-based sterilization procedures. In order to produce delivery vehicles of about 50-200 nm, it is necessary to process the MLV's into uni-lamellar vesicles (ULV's) and thus remove the bulk of particles whose size is greater than about 200 nm. A number of ‘liposome-sizing’ techniques have been developed for conventional high cholesterol-containing delivery vehicles wherein the heterogeneous suspension of MLV's is size-reduced using homogenization, sonication and/or extrusion.

When extruding, the MLV suspension is typically passed through filters with pore sizes of about 0.2 microns or less, at temperatures above phase transition. Particle size determination with techniques such as Dynamic Light Scattering confirms that these extrusion methods produce a suspension of lipid vehicles whose polydispersity has been narrowed to vesicles that are predominantly less than 200 nm (which is necessary for filter sterilization). High cholesterol-containing liposomes that are commonly used in the art are readily extruded at temperatures above the phase transition temperature of the highest melting lipid in the liposomes and the resulting suspension is able to be filter sterilized using standard, known methods. Similarly, low-cholesterol liposomes can be extruded at temperatures above their phase transition temperature and particle sizing demonstrates that the resulting suspension has a size distribution pattern somewhat similar to high cholesterol-containing liposomes. However, contrary to what one would expect, the extruded ‘low-cholesterol’ suspension is considerably more difficult to filter sterilize. The inability to filter sterilize resides in the fact that the filters quickly become ‘clogged’ after the extruded suspension has been applied to the filter, even under high pressure and even though the mean liposome diameter is significantly lower than 0.2 microns.

Because cholesterol that acts to stabilize the lipid membrane is absent, low-cholesterol delivery vehicles are significantly more fluid than their high cholesterol-containing counterparts at temperatures above their phase transition temperature, i.e., the high temperatures conventionally used for extrusion of liposomes. The delivery vehicles are more rigid at low temperatures than at high temperatures and are therefore in the gel- or solid-state when below their phase transition temperature. While not intending to be bound by any theory, it is postulated that due to the increased plasticity of low-cholesterol liposomes at high temperatures, a number of excessively large vesicles are ‘squeezed’ or ‘contorted’ through the extrusion filter. At the lower temperatures used for filter sterilization, these larger liposomes become rigid and are unable to deform. As a result, these large and now rigid liposomes ‘clog’ the 0.2 micron filters during the sterilization process. A need therefore exists to identify liposome-sizing methods that reduce the number of large, low-cholesterol particles that pass through extrusion filters, in order to achieve a less polydisperse suspension which can thus be filter sterilized. New methods will also reduce the unwarranted costs and labor associated with the loss of product and supplies that occurs when filters become clogged. In addition, by eliminating the need to refresh or change filters during sterilization, improved quality and sterility of the final liposomal suspension can be achieved.

It is recognized that the filtration difficulties encountered for low-cholesterol liposomes is likely to arise for any lipid-based delivery vehicle that is similarly ‘rigid’ (i.e., in the gel or solid-state) at temperatures utilized for filter sterilization. These ‘gel-phase’ delivery vehicles may or may not contain low levels of cholesterol depending upon the lipid composition and/or the presence of additional membrane-rigidifying agents. Since many of these agents have properties distinct from cholesterol, their presence may have the same rigidifying affect whether they are formulated at low or high concentrations. It is thus intended that the scope of this invention includes lipid-based delivery vehicles which are substantially in the gel- or solid-state under normal filter sterilization conditions (i.e., below their phase transition temperature), such as low-cholesterol liposomes.

A novel method for reducing the polydispersity of a preparation of gel-phase delivery vehicles (examples comprise low-cholesterol liposomes) such that they are capable of being successfully filter sterilized has now been found. Once a heterogeneous-sized, e.g., MLV preparation has been initially extruded using techniques conventionally used in the art (i.e., at a temperature above the phase transition temperature of the vesicles), the resulting sample is subsequently extruded at a temperature below the phase transition temperature (thus in the gel-state) of the vesicles. At this lower temperature, the gel-phase delivery vehicles are more rigid and therefore less likely to deform and pass through a filter whose size is smaller than the vesicle size. Once the secondary extrusion has taken place, the polydispersity of the sample is reduced by eliminating excessively large particles and the delivery vehicles are effectively filter-sterilized using 0.2 micron filters and conventional filter sterilization techniques.

### DISCLOSURE OF THE INVENTION

The present invention is based on the discovery that lipid-based delivery vehicles that are rigid and non-deformable under normal sterilization conditions are not effectively filter sterilized after utilizing conventional liposome size-reduction techniques, but that these ‘rigid’ or gel-phase delivery vehicles require additional sizing methods to reduce their polydispersity to levels sufficient for filter sterilization. The additional sizing methods required are ideally performed at temperatures below the phase transition temperature of the delivery vehicle.

The invention thus provides a method of reducing the polydispersity of a suspension of gel-phase lipid-based delivery vehicles such that the suspension is capable of being filter sterilized. In one embodiment, the delivery vehicles are MLV's containing low levels of cholesterol or other membrane-rigidifying agent(s). The delivery vehicles may be extruded at least once at a temperature above their phase transition temperature and then at least once at a temperature below their phase transition temperature.

“Gel-phase lipid-based delivery vehicles” are particles composed of lipids that are rigid and non-deformable under normal filter sterilization conditions (i.e., typically at temperatures below their phase transition temperatures) so that they are difficult to pass through conventional filters used in the sterilization process when their dimensions exceed the pore size of the filter.

Thus, in one aspect, the invention is directed to a method to reduce the polydispersity of a suspension of gel-phase lipid-based delivery vehicles which comprises the step of extruding a suspension of said vehicles at a temperature below the phase transition temperature of the vehicles. This may be preceded by extruding the suspension at a temperature above the phase transition temperature of the vehicles.

In another aspect, the invention relates to compositions prepared by the methods of the invention. These compositions are characterized by more uniform size of the delivery vehicles, reduced values of the maximum diameter below which 99% of the vesicles in the suspension fall, and reduced percentages of vehicles that exceed diameters >200 nm.

The invention is useful for compositions which contain polydisperse suspensions of gel-phase lipid-based delivery vehicles and which suspensions contain undesired percentages of such vehicles that are larger than sterilization filtration pore size—i.e., 0.2μ or 200 nm. Typical of such delivery vehicles are liposomes, particularly those containing less than 25 mol % cholesterol or less than 20 mol % cholesterol or less than 10 mol % cholesterol. However, other liposome and lipid-based delivery vehicles may also be employed in the method of the invention and that include undesirable polydispersity or undesirable levels of larger particles due to their lipid composition or mode of preparation.

It should be noted that filtration performed in the context of extrusion is a different process from filtration performed for sterilization. Extrusion through filters involves the use of high pressures which forces the particulates through the filter pores, typically by causing larger size particles to disassemble and in addition by forcing the particles which are of sufficiently small size to penetrate the pores through any barrier created by larger particles. Sterilization filtration, on the other hand, cannot employ sufficient pressures to accomplish this “forced filtration” since doing so would introduce sufficient air or other gaseous contaminants to undermine its purpose. Typically, filtration for sterilization is conducted at low pressures and thus the presence of particles too large to penetrate the pores simply results in clogging the filter.

### BRIEF DESCRIPTION OF THE DRAWINGS

FIG. 1A summarizes the polydispersity and size parameters of a suspension of empty DSPC/DSPG/Cholesterol (70/20/10 mol %) liposomes which were extruded 8 times through 100 nm pore size polycarbonate filters at 70° C. and analyzed using Dynamic Light Scattering (DLS).

FIG. 1B summarizes the polydispersity and size parameters of the same suspension of empty DSPC/DSPG/Cholesterol (70/20/10 mol %) liposomes extruded at 70° C. and used in FIG. 1A but which were then extruded 2 times through 100 nm pore size polycarbonate filters at 40° C. and analyzed using DLS.

### MODES OF CARRYING OUT THE INVENTION

The method of the invention involves size-reducing and reducing polydispersions of a suspension of gel-phase lipid-based delivery vehicles (e.g., low-cholesterol liposomes) such that the suspension can be successfully filter sterilized using standard sterilization techniques. The method may include first size-reducing said delivery vehicles by employing size-reduction techniques commonly used in the art, but, in any event, by extruding a suspension of the vehicles at least once at a temperature below their phase transition temperature prior to filter sterilization

By subjecting a suspension of the delivery vehicles at a temperature below their phase transition temperature to extrusion, the polydispersion of the particles in the suspension may be reduced such that the standard deviation from the mean diameter of the particles is reduced to less than 25% of the mean diameter, preferably less than 20%, more preferably less than 10%. Further, the percentage of vesicles that exceed diameters greater than 200 nm is significantly reduced. The compositions of the invention thus will have reduction in the number of particles larger than 200 nm of 5%, 20%, 30% or 50% by virtue of the method of the invention. Accordingly, the resulting compositions contain such larger particles only at levels of 10%, 5%, 2%, 1% or less. Further, the maximum diameter below which 99% of the vesicles in the suspension fall is reduced by 5%, 10% or 20%. Thus, this maximum diameter is less than 170 nm, or less than 160 nm or less than 150 nm.

Lipid-based delivery vehicles are particulates composed of lipids and may include lipid carriers, liposomes, lipid micelles, lipoprotein micelles, lipid-stabilized emulsions, polymer-lipid hybrid systems, and the like. Liposomes can be prepared as described in Liposomes: Rational Design (A. S. Janoff ed., Marcel Dekker, Inc., N.Y.) or by additional techniques known to those knowledgeable in the art. Liposomes may be prepared to be of “low-cholesterol.” The incorporation of less than 20 mol % cholesterol in liposomes can allow for retention of drugs not optimally retained when liposomes are prepared with greater than 20 mol % cholesterol. Additionally, liposomes prepared with less than 20 mol % cholesterol display narrow phase transition temperatures, a property that may be exploited for the preparation of liposomes that release encapsulated agents due to the application of heat (thermosensitive liposomes). Liposomes of the invention may also contain therapeutic lipids, which include ether lipids, phosphatidic acid, phosphonates, ceramide and ceramide analogues, sphingosine and sphingosine analogues and serine-containing lipids. Liposomes may also be prepared with surface stabilizing hydrophilic polymer-lipid conjugates such as polyethylene glycol-DSPE, to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Cholesterol-free liposomes containing PG or PI to prevent aggregation may be prepared, thereby increasing the blood residence time of the carrier.

Micelles are self-assembling particles composed of amphipathic lipids or polymeric components that are utilized for the delivery of sparingly soluble agents present in the hydrophobic core. Various means for the preparation of micellar delivery vehicles are available and may be carried out with ease by one skilled in the art. For instance, lipid micelles may be prepared as described in Perkins, et al., Int. J. Pharm. (2000) 200(1):27-39. Lipoprotein micelles can be prepared from natural or artificial lipoproteins including low and high-density lipoproteins and chylomicrons. Lipid-stabilized emulsions are micelles prepared such that they comprise an oil filled core stabilized by an emulsifying component such as a monolayer or bilayer of lipids. The core may comprise fatty acid esters such as triacylglycerol (corn oil). The monolayer or bilayer may comprise a hydrophilic polymer lipid conjugate such as DSPE-PEG. These delivery vehicles may be prepared by homogenization of the oil in the presence of the polymer lipid conjugate. Agents that are incorporated into lipid-stabilized emulsions are generally poorly water-soluble. Synthetic polymer analogues that display properties similar to lipoproteins such as micelles of stearic acid esters or poly(ethylene oxide) block-poly(hydroxyethyl-L-aspartamide) and poly(ethylene oxide)-block-poly(hydroxyhexyl-L-aspartamide) may also be used in the practice of this invention (Lavasanifar, et al., J. Biomed. Mater. Res. (2000) 52:831-835).

Preferably, liposomes will be used in the practice of the invention, more preferably, ‘low-cholesterol’ liposomes (comprising less than 25 mol % cholesterol).

Thus, In one embodiment, gel-phase liposomes with reduced polydispersity are generated by initially extruding lipid films at a high temperature (above the liposome phase transition temperature as routinely performed in the art) and the resulting suspension of liposomes are extruded at a lower temperature (below the phase transition temperature of the liposomes). The final, more homogenous, liposomal suspension is then able to be filter sterilized using standard, known techniques.

The term “liposome” as used herein means vesicles comprised of one or more concentrically ordered lipid bilayers encapsulating an aqueous phase. Included in this definition are uni-lamellar vesicles, ULV's. The term “uni-lamellar vesicle” as used herein means single-bilayer vesicles or substantially single-bilayer vesicles encapsulating an aqueous phase wherein the vesicle is less than 500 nm. The uni-lamellar vesicle is preferably a “large uni-lamellar vesicle (LUV)” which is a uni-lamellar vesicle with a diameter between 500 and 50 nm, preferably 200 to 80 nm. As stated above, “gel-phase” refers to lipid-based delivery vehicles which are rigid and non-deformable under normal filter sterilization conditions. Filter sterilization is normally carried out at room temperature which is below the phase transition temperature of most lipid-based delivery vehicles used in the art.

Some of the gel-phase liposomes for use in this invention are prepared to be of “low-cholesterol.” Such liposomes contain an amount of cholesterol that is insufficient to significantly alter the phase transition characteristics of the liposome (typically less than 20 mol %). The incorporation of less than 20 mol % cholesterol in liposomes can allow for retention of drugs not optimally retained when liposomes are prepared with greater than 20 mol %/o of cholesterol or such agents. Additionally, liposomes prepared with less than 20 mol % cholesterol display narrow phase transition temperatures, a property that may be exploited for the preparation of liposomes that release encapsulated agents once administered due to the application of heat (i.e., “thermosensitive liposomes”). ‘Gel-phase’ delivery vehicles may contain a membrane-rigidifying agent(s) aside from cholesterol, such as other sterols. Since many of these agents have properties distinct from cholesterol, their presence may have the same rigidifying affect whether they are formulated at low or high concentrations. The invention includes use of lipid-based delivery vehicles that are rigid or substantially in the gel-phase when below their phase transition temperature, such as low-cholesterol liposomes.

Liposomes of the present invention or for use in the present invention may be generated by a variety of techniques including but not limited to lipid film/hydration, reverse phase evaporation, detergent dialysis, freeze/thaw, homogenization, solvent dilution and extrusion procedures. Preferably, the liposomes are generated by extrusion procedures described by Hope, et al., Biochim. Biophys. Acta (1984) 55-64 and set forth in the Examples below.

Formation of liposomes requires the presence of “vesicle-forming lipids” which are amphipathic lipids capable of either forming or being incorporated into a bilayer structure. The latter term includes lipids that are capable of forming a bilayer by themselves or when in combination with another lipid or lipids. An amphipathic lipid is incorporated into a lipid bilayer by having its hydrophobic moiety in contact with the interior, hydrophobic region of the membrane bilayer and its polar head moiety oriented toward an outer, polar surface of the membrane. Hydrophilicity may arise from the presence of functional groups such as hydroxyl, phosphato, carboxyl, sulfato, amino or sulfhydryl groups. Hydrophobicity results from the presence of a long chain of aliphatic hydrocarbon groups. The vesicle-forming lipids included in the liposomes of the invention will typically comprise at least one acyl group with a chain length of at least 16 carbon atoms. Particularly preferred phospholipids used as vesicle forming components include dipalmitoyl phosphatidylcholine (DPPC) and distearoyl phosphatidylcholine (DSPC).

DPPC is a common saturated chain (C16) phospholipid with a bilayer phase transition temperature of 41.5° C. Liposomes containing DPPC and other lipids that have a similar or higher transition temperature, and that mix ideally with DPPC (such as 1,2-dipalmitoyl-sn-glycero-3-[phospho-rac-(1-glycerol)] (DPPG) (Tc=41.5° C.) and 1,2-distearoyl-sn-glycero-3-phosphocholine (DSPC) (Tc=55.1° C.)) have been studied. Thus, the liposomes of the invention typically have a phase transition temperature greater than 38° C.; this can be assured by employing components which confer this property. The ultimate transition temperature will depend on the acyl chain length as well as the degree of unsaturation of the acyl groups. Typically, including unsaturation in the chain lowers the transition temperature so that in the event the acyl groups are unsaturated, acyl groups containing 18 carbons or 20 carbons or more are preferred.

Liposomes may also be prepared such that the liquid crystalline transition temperature is greater than 45° C. Vesicle-forming lipids making up the liposome are phospholipids such as phosphatidylcholine (PC), phosphatidyl (PA) or phosphatidylethanolamine (PE), containing two saturated fatty acids, within the acyl chains are preferably stearoyl (18:0), nonadecanoyl (19:0), arachidoyl (20:0), heniecosanoyl (21:0), behenoyl (22:0), tricosanoyl (23:0), lignoceroyl (24:0) or cerotoyl (26:0).

The liposomes of the invention comprise amphipathic lipids as vesicle-forming lipids, but reduced amounts of cholesterol. Such lipids include sphingomyelins, glycolipids, ceramides and phospholipids. Such lipids may include lipids having targeting agents, ligands, antibodies or other such components which are used in liposomes, either covalently or non-covalently bound to lipid components.

Liposomes of the invention may contain therapeutic lipids, which include ether lipids, phosphatidic acid, phosphonates, ceramide and ceramide analogues, sphingosine and sphingosine analogues and serine-containing lipids. Liposomes may also be prepared with surface stabilizing hydrophilic polymer-lipid conjugates such as polyethylene glycol-DSPE, to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Embodiments of this invention may make use of low-cholesterol liposomes containing PG to prevent aggregation thereby increasing the blood residence time of the carrier.

Liposomes of the invention may be prepared and size-reduced when “empty” or may contain an encapsulated biologically active agent. By “empty,” it is meant that delivery vehicles contain little to no biological, diagnostic or cosmetic agents. Biologically active agents are typically small molecule drugs useful in treatment of neoplasms or other diseases. The drugs are incorporated into the aqueous internal compartment(s) of the liposomes either by passive or active loading procedures. In passive loading, the biologically active agent is simply included in the preparation from which the liposomes are formed or alternatively, can be passively loaded after the liposomes have been prepared. Active loading procedures can be employed, such as ion gradients, ionophores, pH gradients and metal-based loading-procedures based on metal complexation.

By “loaded” or “encapsulated”, it is meant stable association of the active agent with the delivery vehicle. Thus, it is not necessary for the vehicle to surround the agents as long as the agents are stably associated with the vehicles when administered in vivo. Thus, “stably associated with” and “loaded in” or “loaded with” or “encapsulated in” or “encapsulated with” are intended to be synonymous terms.

A heterodisperse suspension of liposomes formed by methods described above may be ‘size reduced’ using conventional techniques to produce liposomes within a desired size range and reduced polydispersity. Conventional size-reduction techniques include but are not limited to sonication, homogenization and extrusion. Preferably extrusion is used in the practice of the invention. Standard extrusion methods commonly used in the art are advantageous over the former two techniques in that a variety of membrane pore sizes are available to produce liposomes in various size ranges. A drawback of this technique for low-cholesterol liposomes is the tendency for large vesicles to deform and thus pass through narrow extrusion filters when extruded under standard temperatures (i.e., above the vesicle phase transition temperature). The result is a suspension with increased heterogeneity containing oversized vesicles. A farther drawback of this technique is the inability to filter sterilize the resultant suspension. In one embodiment, a heterodisperse suspension of MLV's is extruded at least once at the higher temperature and then at least once at a temperature below the vesicle phase transition temperature, thus overcoming the difficulties in size-reducing gel-phase liposomes to levels that are sufficient for filter sterilization by reducing the number of excessively large liposomes that inadvertently pass through the extrusion filter at high temperatures.

“Above the phase transition temperature” is a temperature above the phase transition temperature of the highest melting lipid in the lipid-based delivery vehicle.

The liposomes of the present invention may be administered to warm-blooded animals, including humans. These liposome and lipid carrier compositions may be used to treat a variety of diseases in warm-blooded animals. Examples of medical uses of the compositions of the present invention include but are not limited to treating cancer, treating cardiovascular diseases such as hypertension, cardiac arrhythmia and restenosis, treating bacterial, fungal or parasitic infections, treating and/or preventing diseases through the use of the compositions of the present inventions as vaccines, treating inflammation or treating autoimmune diseases. For treatment of human ailments, a qualified physician will determine how the compositions of the present invention should be utilized with respect to dose, schedule and route of administration using established protocols. Such applications may also utilize dose escalation should bioactive agents encapsulated in liposomes and lipid carriers of the present invention exhibit reduced toxicity to healthy tissues of the subject.

Pharmaceutical compositions comprising the liposomes of the invention are prepared according to standard techniques and further comprise a pharmaceutically acceptable carrier. Generally, normal saline will be employed as the pharmaceutically acceptable carrier. Other suitable carriers include, for example, water, buffered water, 0.4% saline, 0.3% glycine, and the like, including glycoproteins for enhanced stability, such as albumin, lipoprotein, globulin, etc.

### EXAMPLES

The following examples are given for the purpose of illustration and are not by way of limitation on the scope of the invention.

### Example 1

### The Effect of Extrusion Temperature on Ease of Filtration

A suspension of gel-phase liposomes were extruded either once at a temperature above the liposomal phase transition temperature or at said temperature and then again at a temperature below the liposomal phase transition temperature in order to determine the effect of extrusion temperature on the ease of filtration using a standard 0.2 micron depth filter routinely used in the art for sterilization.

Lipid films of DSPC/DSPG/Cholesterol at a mole ratio of 70:20:10 were prepared by dissolving lipids in chloroform:methanol:water (95:4:1 vol/vol/vol) and subsequently dried under a stream of nitrogen gas and placed in a vacuum pump to remove solvent. Lipid levels were quantified during the formulation process using High Performance Liquid Chromatography. The resulting lipid film was placed under high vacuum for a minimum of 2 hours. The lipid film was hydrated in 100 mM Cu(II)gluconate adjusted to pH 7.4 with triethanolamine (TEA) to form multi-lamellar vesicles (MLV's). The resulting preparation was extruded 8 times through stacked 100 nm polycarbonate filters at 70 (above the liposomal phase transition temperature) and then cooled to room temperature and applied to a 25 mm, 0.2 micron filter using a 20 mL syringe. The sample was extremely viscous and required large amounts of pressure of pass through the filter. On average, less than 25 milliliters of sample was able to pass through the sterilization filter before it became clogged and unusable.

In order to determine whether an excess of large, gel-phase liposomes had unwittingly passed through the extrusion filter, the lipid preparation as described above was similarly extruded 8 times at 70° C. and then subsequently extruded 2 times through stacked 100 nm polycarbonate filters at 40° C. (below the liposomal phase transition temperature). The resulting sample was then passed through an identical 0.2 micron sterilization filter using a 20 mL syringe after being cooled to room temperature. The pressure required to pass the sample through the filter was significantly less than that required when the 40° C. extrusion did not take place. Also, importantly, on average more than 45 milliliters of sample passed through the sterilization filter before it became clogged.

### Example 2

### The Effect of Extrusion Temperature on Size Parameters

Dynamic Light Scattering was used to analyze the size parameters of a suspension of gel-phase liposomes which had been extruded once at a temperature above the liposomal phase transition temperature and subsequently at a temperature below the phase transition temperature in order to determine the effect of extrusion temperature on both the mean particle size and polydispersity of gel-phase liposomes.

Lipid films of DSPC/DSPG/Cholesterol at a mole ratio of 70:20:10 were prepared by dissolving lipids in chloroform:methanol:water (95:4:1 vol/vol/vol) and subsequently dried under a stream of nitrogen gas and placed in a vacuum pump to remove solvent. Lipid levels were quantified during the formulation process using High Performance Liquid Chromatography. The resulting lipid film was placed under high vacuum for a minimum of 2 hours. The lipid film was hydrated in 100 mM Cu(II)gluconate adjusted to pH 7.4 with triethanolamine (TEA) to form multi-lamellar vesicles (MLV's). The resulting preparation was extruded 8 times through stacked 100 nm polycarbonate filters at 70° C. and the mean liposome size as well as polydispersity was analyzed using a NiComp Particle Sizing System (Santa Barbara, Calif.). The printouts shown in the following drawings detail the “Mean Diameter,” “Standard Deviation” and “99% of distribution<” Cumulative Result among others. The “Mean Diameter” as listed under the Gaussian Summary gives the mean vesicle diameter detected in the suspension. Since a 100 nm filter was used for extrusion, the mean diameter should be approximately 100 nm. The “Standard Deviation” listed below the Mean Diameter (abbreviated as “Stnd. Deviation”) represents the deviation in size from the mean vesicle diameter and therefore a larger standard deviation indicates a wider distribution of sizes (or wider bell curve) which would include an increased number of both large and small vesicles. The “99% of distribution” in the Cumulative Result section indicates that 99% of the vesicles in the sample are smaller in size than the value given. This measurement aids in identifying the number of excessively large vesicles found in the sample. Ideally the 99% of distribution value is less than 200 nm and as close to 100 nm as possible since 100 nm extrusion filters were utilized.

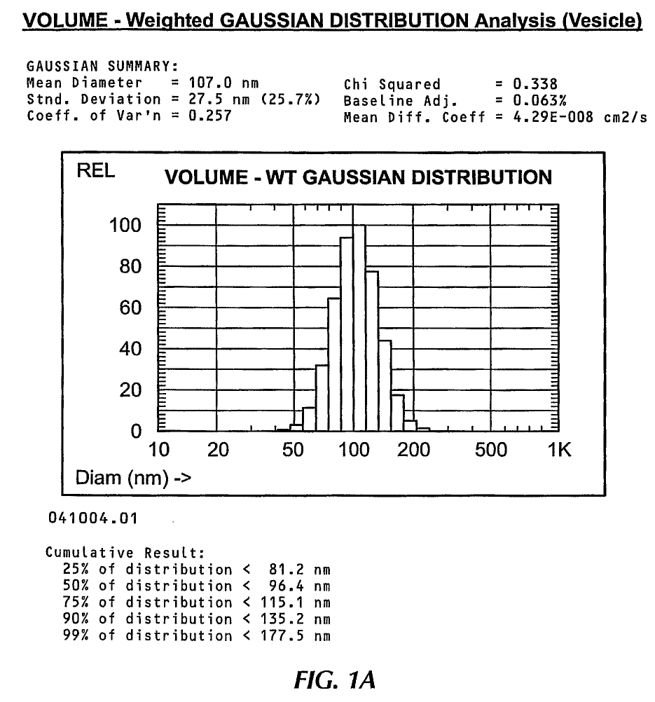

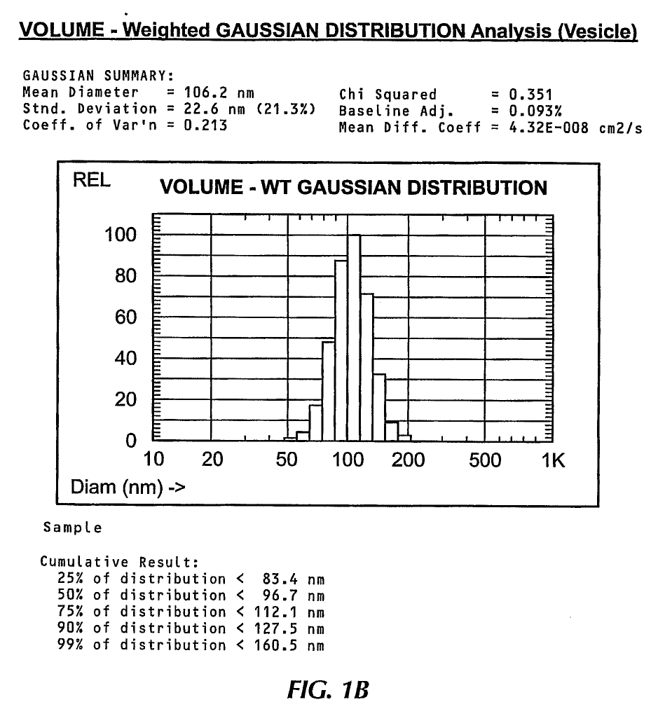

Results summarized in FIG. 1A show that a sample of gel-phase liposomes which were extruded 8 times at 70° C. have a mean diameter of 107.0 nm and a standard deviation of 27.5. The 99% of distribution value indicates that 99% of the vesicles in the sample are less than 177.5 nm in diameter.

A second liposome sample was extruded at 70° C. as described above and then further extruded 2 times through stacked 100 nm polycarbonate filters at 40° C., which is below the liposomal phase transition temperature. The mean liposome size as well as polydispersity was analyzed as previously detailed using a NiComp Particle Sizing System. Since both extrusion methods used a 100 nm filter, the mean diameter is not expected to deviate significantly from the results in FIG. 1A. Results summarized in FIG. 1B show that after the subsequent extrusion at 40° C. the mean diameter, as expected, did not significantly change (106.2 nm). However, the standard deviation was reduced to 22.6 and the 99% of distribution indicates that 99% of the vesicles in the sample now have a diameter less than 160.5 nm.

The results above clearly show that the additional extrusion at 40° C. acted to reduce the number of large liposomes. These findings suggest that the increased ease of filtration after the 40° C. extrusion as outlined in Example 1 was a result of reducing the number of large liposomes that are capable of clogging the sterilization filters.

1. A method for reducing the polydispersity of a suspension of gel-phase lipid-based delivery vehicles which method comprises the step of extruding a suspension of said vehicles at a temperature below the phase transition temperature of said vehicles.

2. The method of claim 1, which further comprises, prior to said step, extruding said suspension at a temperature above the phase transition temperature of said vehicles.

3. The method of claim 1 or 2 wherein the delivery vehicles are liposomes.

4. The method of claim 3 wherein the liposomes comprise a membrane-rigidifying agent.

5. The method of claim 4 wherein the membrane-rigidifying agent comprises cholesterol.

6. The method of claim 3 wherein the liposomes comprise less than 25 mol % cholesterol.

7. A composition of gel-phase lipid-based delivery vehicles prepared by the methods of any of claims 1-6.

8. A composition of gel-phase lipid-based delivery vehicles that is sufficiently size-controlled that a suspension of said vehicles can be effectively filter sterilized.

9. The composition of claim 8 wherein the vehicles are liposomes.

10. The composition of claim 9 wherein the liposomes comprise a membrane-rigidifying agent.

11. The composition of claim 10 wherein the membrane-rigidifying agent comprises cholesterol.

12. The composition of claim 9 wherein the liposomes comprise less than 25 mol % cholesterol.
